(19) United States
(12) Patent Application Publication
(10) Pub. No.: US 2020/0405299 A1
(43) Pub. Date: Dec. 31, 2020

(54) CHIRAL NEEDLE PACKAGES AND RECEPTACLES

(71) Applicant: SHARP FLUIDICS, LLC, Hayward, CA (US)

(72) Inventors: Kenneth B. TRAUNER, San Francisco, CA (US); Douglas G. RIMER, Los Altos Hills, CA (US); Josef E. GOREK, Ross, CA (US)

(73) Assignee: SHARP FLUIDICS, LLC, Hayward, CA (US)

(21) Appl. No.: 15/733,570

(22) PCT Filed: Mar. 6, 2019
(86) PCT No.: PCT/US19/20972

§ 371 Date: Aug. 28, 2020

Related U.S. Application Data
(60) Provisional application No. 62/639,478, filed on Mar. 6, 2018.

(51) Int. Cl. A61B 17/06; A61B 90/00

(52) U.S. Cl. CPC A61B 17/06114; A61B 2017/00455; A61B 90/39

(57) ABSTRACT

A chiral needle package and receptacle are configured together so as to allow a user to configure the suture package and receptacle in a first chiral configuration for right handed use and a second chiral configuration for left handed use. The chirality configurable suture package and needle receptacle can be configured in many ways, and may comprise a first cover on a first side to allow access to the one or more needles and the needle receptacle on first side corresponding to right handed use and chirality, and a second cover on a second side corresponding to left handed use and chirality. The chirality configurable suture package can be packaged with the chirality configurable needle receptacle in order to dispense and to receive needles, such that the suture package and the needle trap are packaged together in a sterile housing configured for right handed use or left-handed use.

### CROSS-REFERENCE TO RELATED APPLICATIONS

The present PCT application claims priority to U.S. Provisional Patent Application No. 62/639,478, filed on Mar. 6, 2018, entitled “CHIRAL NEEDLE PACKAGES AND RECEPTACLES”, the entire disclosure of which is incorporated herein by reference.

### BACKGROUND

Prior method and apparatus of handling suture packages and needle receptacles can be less than ideal in at least some respects. For example, suture needles are typically curved, and proper handling of these suture needles can rely on the user grasping the needle with a needle driver such that the tip of the needle is oriented appropriately for the user to drive the needle into tissue. With some prior approaches an assistant can grasp a needle with a needle driver so that it is properly oriented for the user. The manner in which the needle driver is oriented with respect to the suture needle can change depending on whether the user (e.g. a surgeon), is left or right handed. Prior suture packs can present needles in suture packages with less than ideal chirality, and in at least some instances the assistant or the user (e.g. surgeon) can be required to reorient the needle in the needle driver prior to using the needle to suture a patient. In at least some instances, a surgeon or other medical staff who uses suture needles on a patient may need to reorient the needle after being grasped with a needle driver, which can be particularly challenging for a user who is operating in a near surgical field. For example, in the near surgical field the surgeon will often have several other critical tasks to perform. Also, in the near surgical field bodily fluids and tissue can be present on protective garments, and penetration of protective wear such as gloves can create an elevated risk of infection.

Prior needle receptacles can be less than ideal in at least some respects. For example, the use of prior needle receptacles can be less than ideally suited to address the chirality of the user and the associated needle receptacle, for example. This can result in the user engaging in additional manipulations and can impede workflow of the user in securing and protecting sharps such as needles in at least some instances.

In light of the above, it would be desirable to have improved methods and apparatus for dispensing sharps such as suture needles and securing sharp objects once dispensed. Ideally, such methods and apparatus will overcome at least some of the aforementioned shortcomings of the prior art.

### SUMMARY

The methods and apparatus disclosed herein can decrease manipulations associated with dispensing and securing sharp objects such as suture needles when grasped with a tool such as a needle driver. The suture needle can be accessible to a needle driver in a chiral configuration and a needle receptacle configured to receive the needle in the chiral configuration, so as to decrease manipulations by the user, which can make the needle package and receptacle easier to use, faster and safer. In some embodiments, the chiral needle package and receptacle are configured together so as to allow a user to configure the suture package and receptacle in a first chiral configuration for right handed use and a second chiral configuration for left handed use. The chirality configurable suture package and needle receptacle can be configured in many ways, and may comprise a first cover on a first side to allow access to the one or more needles and the needle receptacle on first side corresponding to right handed use and chirality, and a second cover on a second side corresponding to left handed use and chirality. The chirality configurable suture package can be packaged with the chirality configurable needle receptacle in order to dispense and to receive needles, such that the suture package and the needle trap are packaged together in a sterile housing configured for right handed use or left-handed use. In some embodiments a cover is removed from a first side of the sterile package to allow access to the needle(s) and the needle receptacle with a first chiral configuration (e.g. right handed), or the cover is removed from a second side of the package to allow access to the needle(s) and the receptacle in a second chiral configuration (e.g. left handed). The sutures and associated support structures can be enclosed within the same sterile containers as the sterile needle receptacle prior to removing a cover. Alternatively or in combination, the needle(s) and the receptacle of used needles can be supported on a shared support structure, which can be flipped to and placed on a backing to allow access to the needles and trap with an appropriate orientation.

According to an aspect of the present disclosure, an apparatus can comprise: a needle dispenser configured to dispense one or more curved needles; a needle receptacle to receive the one or more curved needles; a support coupled to the needle receptacle and the needle dispenser; and a housing to contain a plurality of curved suture needles in a sterile configuration. The housing can comprise a removable cover on at least a first side of the suture package. One or more of the support or the housing can be configured to allow the needle to be grasped in a first chirality corresponding to right handed use or a second chirality corresponding to left handed use. The receptacle can be configured to receive the needle with the needle dispenser in the first configuration or the second configuration.

According to another aspect of the present disclosure, a chirality [symbol] can comprise: a housing to contain a plurality of curved suture needles in a sterile configuration, the housing comprising a removable cover on at least a first side of the suture package; and a support to support the plurality of needles within the housing. One or more of the support or the housing can be configured to allow the needles to be grasped in a first chirality corresponding to right handed use or a second chirality corresponding to left handed use.

In some embodiments, the housing can comprise a first removable cover on a first side for right handed access to the plurality of needles and the needle receptacle, and a second removable cover on a second side for left handed access to the plurality of needles and the needle receptacle. In some cases, the second side can be opposite the first side.

In some embodiments, the support can be configured to move relative to the housing to rotate the needle from a first orientation corresponding to right handed access to a second orientation corresponding to left handed access. Optionally, the needle receptacle may comprise a substantially symmetric configuration to allow left handed use or right-handed use when the needle comprises the first orientation or the second orientation.

In some embodiments, the suture package can comprise a clip configured to hold a suture needle. The clip can be configured to fold outward from the support to allow the needle to be grasped and removed from the clip and subsequently placed in the clip. The clip can also be configured to fold inward toward the support to secure the needle. Optionally, the clip can be folded outward toward a first side for right handed use or rotated outward toward a second side to allow for left handed use.

In some embodiments, the support can comprise a first holding structure to hold a curved needle toward a sharp tip end and a second holding structure to hold the needle toward a tailing suture end. The support can also comprise an engagement structure to engage the needle between the first holding structure and the second holding structure in order to rotate the needle about an axis extending between the first holding structure and the second holding structure, so as to change an orientation of the curved suture needle from a first orientation from for right handed use to a second orientation for left handed use, or vice versa.

In some embodiments, the apparatus or the package can further comprise a needle receptacle, the needle receptacle coupled to the suture package and configured to receive the suture needles when the support or the housing comprises the first configuration for right handed use or the second configuration for left handed use.

In some embodiments, the suture package and the needle recept [symbol] the housing and supported together. The housing can comprise a first removable cover on a first side for right handed access to the plurality of needles and the needle receptacle, and a second removable cover on a second side for left handed access to the plurality of needles and the needle receptacle. The second side can be opposite the first side.

In some embodiments, the needle receptacle can comprise an entry zone and a needle slot to receive the needle. The entry zone can be sized larger than the needle slot to guide the needle into the needle slot. The needle receptacle can comprise a needle driver slot to receive the needle driver and allow the needle driver to advance the needle along the entry zone and into the slot.

In some embodiments, the plurality of needles can be arranged in an array with a sharp end of each of the plurality of needles extending into a support material. Optionally, the support material can comprise foam. Optionally, each of the plurality of needles can be oriented with a sharp end and a tailing suture end facing away from the needle receptacle, with a middle portion of each of the plurality of needles facing toward the needle receptacle. In some alternative cases, the middle portion of each of the plurality of needles can face away from the needle receptacle.

In some embodiments, the needle dispenser and the needle receptacle can be stored in a shared compartment within the sterile housing. The needle dispenser and needle receptacle can be accessed from a same side. Optionally, a cover can be removed to expose the compartment to access the needle dispenser and the needle receptacle from the same side. Optionally, the cover can comprise a single cover.

A further aspect of the present disclosure is directed to a method comprising, determining whether a surgeon is left or right handed and configuring a suture dispensing package and a needle receptacle for the handedness of the surgeon.

In some embodiments, the right-handed orientation may correspond to a clockwise orientation of the needle when accessed by the needle driver, and the right-handed orientation may correspond to a counter clockwise orientation of the needle driver when accessed by the needle driver. An orientation may correspond to a direction the needle is pointing with the sharp tip about an axis of rotation corresponding to a center of a radius of curvature of the curved needle. With the right-handed clockwise orientation, the needle tip can point in a clockwise direction about the center of rotation if the needle is advanced with the tip moving forward along a circumferential path defined by a center of rotation of the needle. With the left handed counter clockwise orientation, the needle tip can point in a counter clockwise direction about the center of rotation as the needle if the needle advanced with the tip moving forward along a circumferential p [symbol] curvature of the needle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a schematic of a suture needle package in a first chiral configuration for right handed use;

FIG. 1B illustrates a schematic of a suture needle package in a second chiral configuration for left handed use in accordance with some embodiments;

FIG. 2A illustrates a perspective view of a suture needle package in a chiral configuration for left handed use in accordance with some embodiments;

FIG. 2B illustrates a sectional view of the suture needle package of FIG. 2A comprising a front cover on a first side to allow access to needles corresponding to right handed use, and a back cover on a second side to allow access to needles corresponding to left handed use in accordance with some embodiments;

FIG. 3A illustrates a schematic exploded view of the front and back covers, with the suture needle package in a first chiral configuration for right handed use and the front cover facing up in accordance with some embodiments;

FIG. 3B illustrates a schematic exploded view of the front and back covers, with the suture needle package in a second chiral configuration for left handed use and the back cover facing up in accordance with some embodiments;

FIGS. 4A, 4B and 4C illustrate a suture needle package comprising an actuation mechanism for switching (rotating) a needle between a first chiral configuration and a second chiral configuration in accordance with some embodiments;

FIGS. 5A and 5B illustrate a suture needle package comprising [symbol] configured to receive a needle driver for advancement of a needle into the slot in accordance with some embodiments;

FIGS. 5C and 5D illustrate a back cover comprising a padding that forms a confluent surface with a trap padding of the housing post assembly in accordance with some embodiments;

FIG. 6A illustrates adhesive covers on both sides of a suture needle package in accordance with some embodiments;

FIG. 6B illustrates the suture needle package of FIG. 6A with an adhesive cover removed.

FIG. 7 illustrates a suture needle package comprising a linkage mechanism that is configured to simultaneously switch (rotate) a plurality of needles between a first chiral configuration and a second chiral configuration in accordance with some embodiments;

FIG. 8A illustrates a suture needle package comprising a plurality of needles in a first chiral configuration for right-handed use, and FIG. 8B shows the plurality of needles rotated to a second chiral configuration for left-handed use in accordance with some embodiments;

FIG. 9 illustrates a suture needle package comprising a plurality of needles that are configured for use from a user's perspective, and comprising one or more suture receiving channels in accordance with some embodiments;

FIG. 10A illustrates a suture needle package with clips for holding suture needles in a secure configuration in accordance with some embodiments;

FIGS. 10B and 10C illustrate the suture needle package of FIG. 10B with a clip in a deployment configuration in accordance with some embodiments;

FIG. 11A illustrates a suture needle package with clips for holding suture needles in a secure configuration, and comprising needle drive slots in accordance with some embodiments;

FIG. 11B illustrates the suture needle package of FIG. 11A with a clip in a deployment configuration with a needle drive slot exposed, in accordance with some embodiments;

FIG. 12 illustrates a suture needle being removed from a suture needle package, in accordance with some embodiments;

FIG. 13A illustrates a suture needle being advanced into a working surface, in accordance with some embodiments;

FIG. 13B illustrates a sharp end of a suture needle protruding from the working surface, in accordance with some embodiments;

FIG. 13C illustrates the suture needle being removed from the working surface, in accordance with some embodiments;

FIG. 14A illustrates the suture needle being inserted into a needl [symbol] with some embodiments;

FIG. 14B illustrates the suture needle being secured in a needle receptacle, in accordance with some embodiments; and

FIGS. 15A and 15B show an orientation of chiral suture needles in a needle package and the orientation placed in a needle receptacle, in accordance with some embodiments.

### DETAILED DESCRIPTION

The present invention is directed towards systems and methods for improving the efficiency of operating rooms. The embodiments disclosed herein are well suited for combination with many prior systems and methods, such as prior suture packs, prior needle holders, and prior operating rooms and personnel.

One approach for improving operating room efficiency is to reduce the manipulations of suture needles in an operating room. The embodiments disclosed herein can facilitate a user's (e.g. surgeon's) manipulation of suture needles, by providing chiral suture packages that can be conveniently used by both right handed and left-handed users, without requiring the users to reorient the needle in a needle driver during use. The chiral suture packages can include various configurations that permit the needle driver to be properly oriented with respect to the suture needle, for both right handed and left-handed users.

While preferred embodiments of the present invention have been shown and described herein, it will be appreciated by those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

It should be noted that the accompanying figures are intended to illustrate the general characteristics of methods and materials with reference to certain example embodiments of the invention and thereby supplement the detailed written description provided below. These drawings are not, however, to scale and may not precisely reflect the characteristics of any given embodiment, and should not be interpreted as defining or limiting the range of values or properties of embodiments within the scope of this invention. In particular, the relative sizing and positioning of particular elements and structures may be reduced or exaggerated for c [symbol] identical reference numbers in the various drawings is intended to indicate the presence of a similar or identical element or feature.

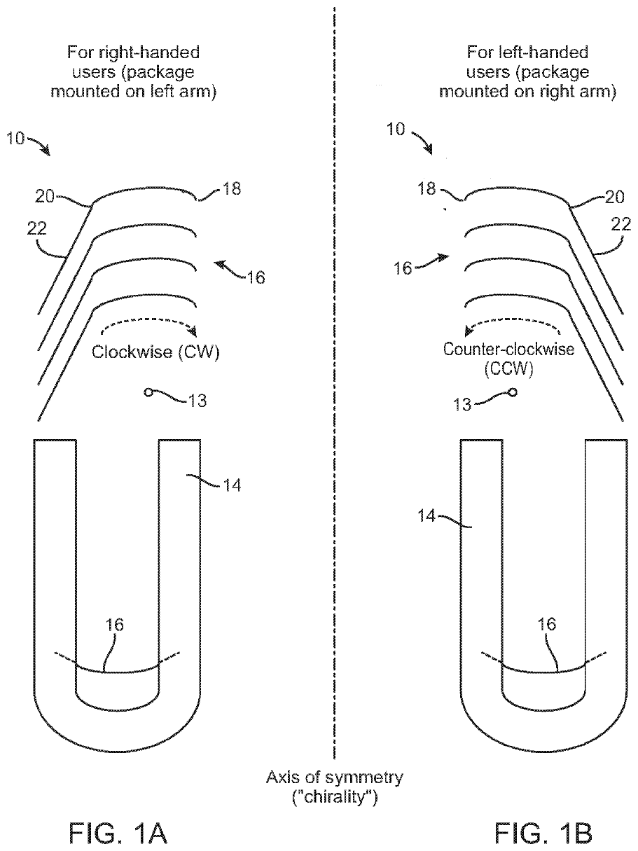

FIGS. 1A and 1B illustrate schematics of a chirality configurable suture package 10 in accordance with some embodiments. The suture package 10 can be configured for both left-handed and right-handed use. For example, FIG. 1A shows a schematic of the suture needle package 10 in a first chiral configuration for right handed use, and FIG. 1B shows a schematic of the suture needle package 10 in a second chiral configuration for left handed use. The dotted line shows an axis of symmetry 12 (“chirality”) for the two chiral configurations.

Referring to FIG. 1A, the suture package 10 may comprise a needle receptacle 14 and a plurality of suture needles 16. A needle 16 may comprise a curved needle having a sharp tip 18 and a tail portions 20. The curved needle 16 may extend from the tip 18 to the tail portions 20. A suture 22 can be coupled to the tail portion 20 of the needle 16. The suture package 10 may be mounted on a left forearm of a user, with the tips 18 of the needles 16 extending away from the left forearm of the user. As shown in FIG. 1A, the needles 16 may be configured to move in a clockwise (CW) orientation about a center of reference axis 13 for right handed use (for right handed users). A needle receptacle 14 may be coupled to the suture package 10 and configured to receive the suture needles 16 when the needles are in the first chiral configuration for right handed use.

Referring to FIG. 1B, the suture package 10 may be flipped relative to the axis of symmetry 12 such that the needles 16 are configured to move in a counter-clockwise (CCW) orientation about a center of reference axis for left handed use (for left handed users). The suture package 10 may be mounted on a right forearm of a user, with the tips 18 of the needles 16 extending away from the right forearm of the user. The needle receptacle 14 may be coupled to the suture package 10 and configured to receive the suture needles 16 when the needles are in the second chiral configuration for left handed use.

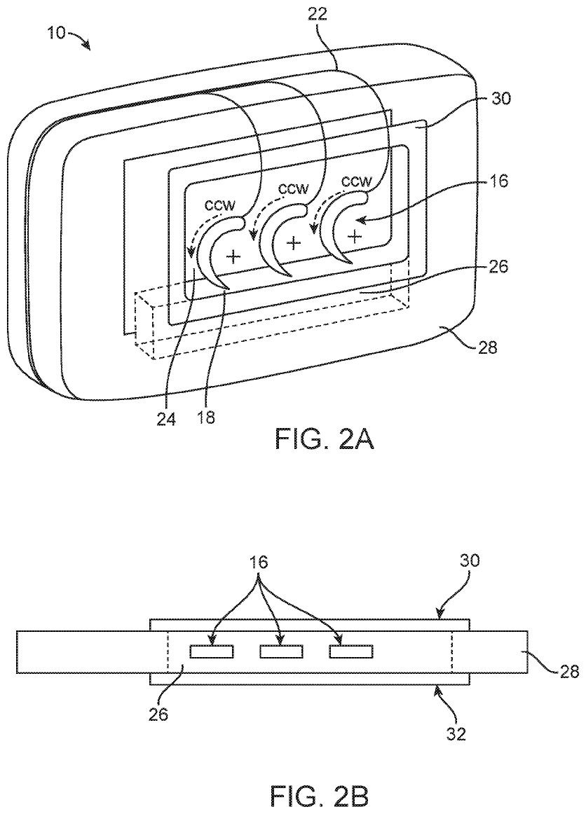

FIG. 2A illustrates a perspective view of a suture needle package 10 in a chiral configuration for left handed use. The suture needle package 10 in FIG. 2A and in any of the embodiments disclosed herein may be referred to interchangeably as “an apparatus.” The apparatus may comprise a needle dispenser 24 configured to dispense one or more curved needles 16, a needle receptacle (not shown) to receive the one or more curved needles 16, a support 26 coupled to the needle receptacle and the needle dispenser 24, and a housing 28 to contain a plurality of curved suture needles 16 in a sterile configuration. The housing 28 may comprise a removable cover 30 on at least a first side of the suture package 10. One or more of the support 26 or the housing 28 can be configured to allow the needle 16 to be grasped in a first chirality corresponding to right ha [symbol] chirality corresponding to left handed use. The receptacle 14 can be configured to receive the needle with the needle dispenser 24 in the first configuration or the second configuration. The needle receptacle 14 can be coupled to the suture package 10 and configured to receive the suture needles 16 when the support 26 or the housing 28 is in (i) the first configuration for right handed use or (ii) the second configuration for left handed use. In some embodiments, the suture package 10 and the needle receptacle 14 can be contained within the housing 28 and supported together.

Needle packages and receptacles suitable for incorporation and modification in accordance with the present disclosure are described in PCT/US2015/027659, filed Apr. 24, 2015, entitled “SYSTEMS AND METHODS FOR INCREASED OPERATING ROOM EFFICIENCY”, and PCT/US2016/059599, filed Oct. 28, 2016, entitled “SYSTEMS AND METHODS FOR INCREASED OPERATING ROOM EFFICIENCY”, the entire disclosures of which are incorporated herein by reference.

The one or more curved needles 16 may be provided as an array of needles, for example as shown in FIG. 2A. The needles 16 may be provided on the support 26 which can be made of a soft or spongy material. In some embodiments, the support 26 may comprise foam and the needles 16 can be embedded within the foam. In the example of FIG. 2A, each of the needles 16 may be configured to be dispensed or manipulated in a counter-clockwise (CCW) orientation relative to a reference axis for left handed use (for left handed users). The needles 16 may be protected by a removable back cover 32 prior to use. The back cover 32 may include an adhesive film or layer. The support 26 may include a support material. In some embodiments, the plurality of needles 16 can be arranged in an array with a sharp end 18 of each of the plurality of needles extending into the support material. The support material can comprise foam.

FIG. 2B illustrates a sectional view of the suture needle package 10 of FIG. 2A comprising a front cover 30 on a first side to allow access to needles 16 corresponding to right handed use, and a back cover 32 on a second side to allow access to needles 16 corresponding to left handed use. The needles 16 can be accessed from either the first side or the second side, depending on whether it is intended for right handed or left-handed use. The housing 28 can comprise a first removable cover 30 (front cover) on a first side for right handed access to the plurality of needles 16 and the needle receptacle 14. The housing 28 can also comprise a second removable cover 32 (back cover) on a second side for left handed access to the plurality of needles 16 and the needle dispenser 24. The second side may be opposite the first side. Accordingly, the front cover 30 and back cover 32 may be opposite to each other, with the support 26 and housing 28 disposed therebetween as shown in FIG. 2B. The first and second removable covers 30, 32 may include an [symbol] attaches the covers 30, 32 to the respective first and second sides. The adhesive material may be configured such that the first and second removable covers 30, 32 are removable (e.g. peelable) by hand. The covers 30, 32 can be peeled off from either side. Removal of the front cover 30 can permit access to the needles 16 for dispensing or manipulation in a clockwise (CW) orientation relative to a reference axis for right handed use (for right handed users). Conversely, removal of the back cover 32 can permit access to the needles 16 for dispensing or manipulation in a counter-clockwise (CCW) orientation relative to a reference axis for left handed use (for left handed users).

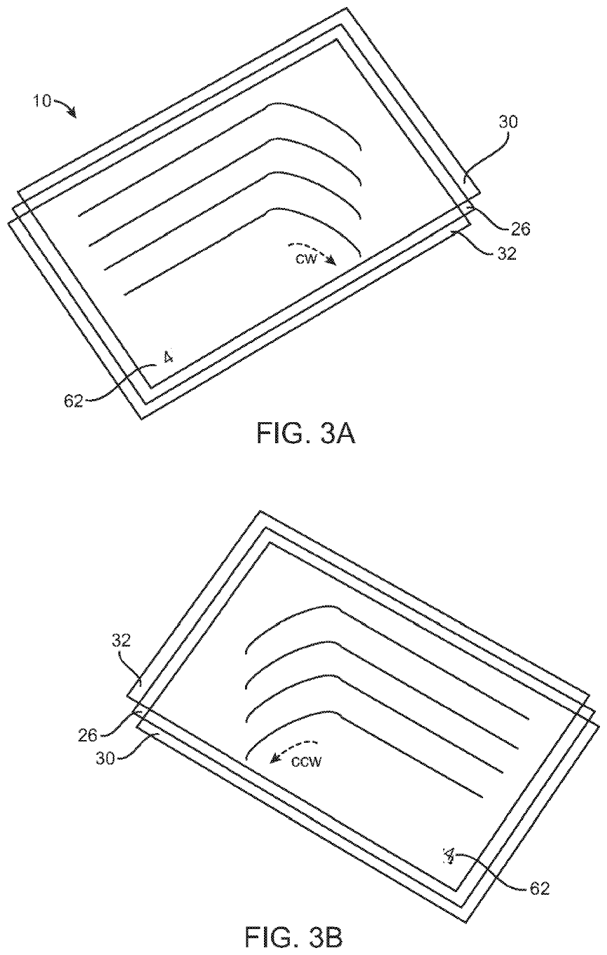

FIG. 3A illustrates a schematic exploded view of the front and back covers 30, 32, with the suture needle package 10 in a first chiral configuration for right handed use and the front cover 30 facing up. The one or more curved needles 16 may be provided as an array of needles on the support ‘26’ in a clockwise (CW) orientation relative for right handed use. Conversely, FIG. 3B illustrates a schematic exploded view of the back and front covers 30, 32 with the suture needle package 10 in a second chiral configuration for left handed use and the back cover 32 facing up. The same one or more curved needles 16 may be provided as an array of needles on the support ‘26’ but in a counter-clockwise (CCW) orientation relative for left handed use. The suture needle package 10 may be flipped relative to the axis of symmetry 12 shown in FIGS. 1A and 1B, to enable the suture package 10 to be used in either the first chiral configuration or the second chiral configuration.

In some embodiment, the suture needle package 10 can have one or markers 62 indicating a quantity of needles 16 contained in the suture needle package 10. This allows the surgeon or assistant to easily reconcile the number of needles initially contained in the suture needle package with the number of needles in the receptacle and the number of the needles remaining in the package, in order to determine if any needles are missing. For example, in the embodiment of FIG. 3A, the one or more markers 62 may be printed on the housing 28 as the number “4”. In some embodiments, the one or more markers 62 may be obscured by the removable covers 30, 32 such that they are not visible when the covers 30, 32 are in place. For example, the one or more markers 62 may be printed on the housing 28 beneath the covers 30, 32. Removal of a cover 30, 32 would then expose a marker 62 rendering it visible. In some embodiments a first marker 62 may be printed on a first side of the housing 28 and a second marker 62 may be printed on a second side of the housing 28. Removal of the front cover 30 may expose the first marker and removal of the back cover 32 may expose the second marker. Alternatively or in combination, the cover may comprise a transparent sterile barrier material that allows the marker to be seen by a user prior to removal of the cover. The cover may comprise a transparent sterile barrier material over a portion of the cover to allow visualization of the marker prior to removal of the cover, or the c [symbol] obscuring sterile barrier material that protects and maintains sterility of the needles in the suture package, but does not extend over the marker.

In some embodiments, the support 26 can be configured to move relative to the housing 28 to rotate one or more needles 16 from a first orientation corresponding to right handed access to a second orientation corresponding to left handed access. In some embodiments, the needle receptacle 14 can comprise a substantially symmetric configuration to allow left handed use or right-handed use when the one or more needles 16 comprises the first orientation or the second orientation.

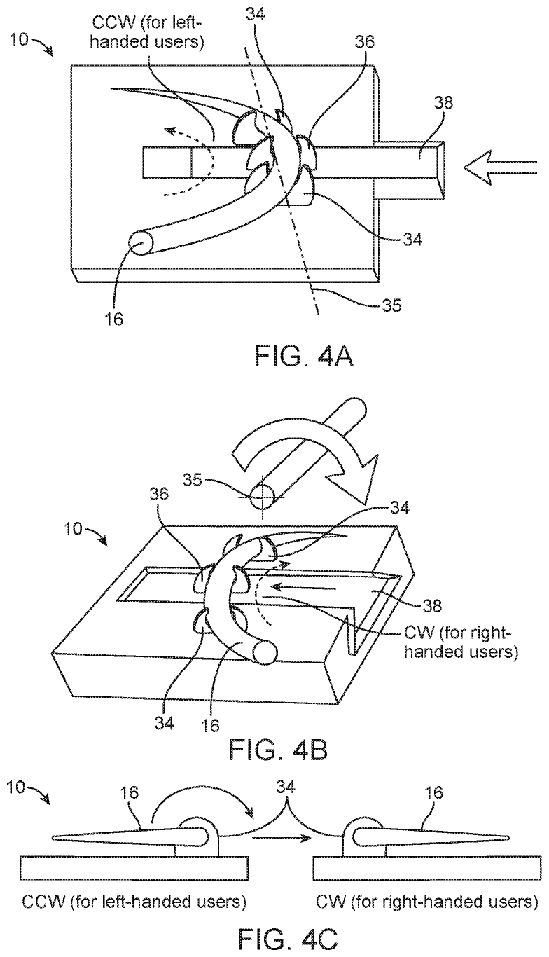

FIGS. 4A, 4C and 4C illustrate a suture needle package 10 comprising an actuation mechanism for switching (rotating) a needle between a first chiral configuration and a second chiral configuration in accordance with some embodiments. The actuation mechanism may comprise a plurality of elements, for example one or more holding structures 34, an engagement structure 36, and an engagement member 38. The holding structures 34 may include one or more protruding structures, e.g. a first protruding structure and a second protruding structure as shown in FIG. 4A. The support 26 can comprise a first holding structure 34 to hold a curved needle 16 toward a sharp tip end 18, and a second holding structure 34 to hold the needle 16 toward a tailing suture end 20. The support 26 can further comprise an engagement structure 36 to engage the needle 16 between the first holding structure 34 and the second holding structure 34, in order to rotate the needle 16 about an axis 35 extending between the first holding structure 34 and the second holding structure 34. Rotation of the needle 16 about the axis can cause a change in orientation of the curved suture needle 16, e.g. from a first orientation for right handed use to a second orientation for left handed use, or vice versa.

Referring to FIG. 4A, the needle 16 may be initially in the second orientation (counter-clockwise CCW) for left handed use. When the engagement structure 36 is translated in the direction as shown (FIG. 4A), the engagement structure 36 engages the needle 16 between the first holding structure 34 and the second holding structure 34, and cause the needle 16 to rotate in a clockwise direction relative to the axis of rotation. The needle 16 is rotated from the second orientation (counter-clockwise CCW) for left handed use to the first orientation (clockwise CW) for right handed use, as illustrated in FIGS. 4B and 4C. Accordingly, the actuation mechanism can be used to switch (rotate) the needle 16 between different chiral configurations, depending on whether the suture package 10 is intended for left handed use or right-handed use.

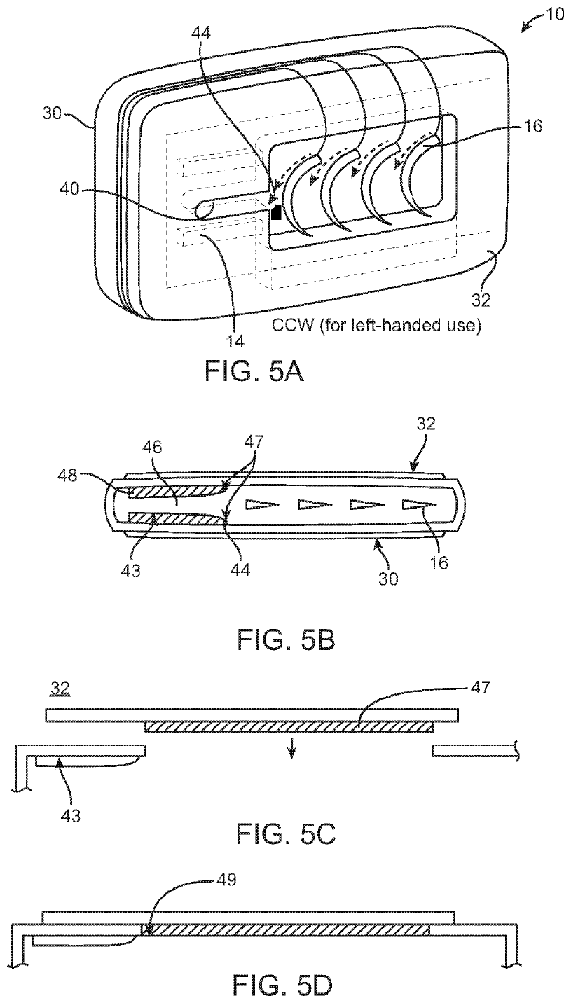

FIGS. 5A and 5B illustrate a suture needle package 10 comprising a needle driver slot 40 configured to receive a needle driver (not shown) for advancement of a needle 16 into the slot 40 in accordance with some embodiments. Referring to FIG. 5A, the needles [symbol] second orientation (counter-clockwise CCW) for left handed use, and protected by the back cover 32. The needle receptacle 14 can comprise an entry zone 44 and a needle slot 46 to receive the needle 16. The entry zone 44 can be sized larger than the needle slot 46 to guide the needle 16 into the needle slot 46. The needle receptacle 14 can comprise a needle driver slot 40 to receive the needle driver and allow the needle driver to advance the needle 16 along the entry zone 44 and into the slot 46. The entry zone 44 may include a beveled entrance to facilitate the receiving of the needle driver 42. The needle slot 46 may correspond to a secure zone 48 for securing the needle 16 therein. The secure zone 48 may include a trap padding 43 for trapping the needle 16 in the secure zone 48.

In some embodiments, the back cover 32 (and/or the front cover 30) may include a padding 47. The padding 47 may be provided on an inner portion of the cover 30, 32 such that the padding 47 comes into contact with the needles 16 when the cover 30, 32 is placed onto the needles 16. The padding 47 may be made of a soft material, for example foam. In some embodiments, the padding 47 on the cover 32 (e.g. back cover) and the trap padding 43 in the secure zone 48 of the needle receptacle 14 may form a confluent surface 49 when the cover 32 is assembled onto the package, for example as shown in FIG. 5D.

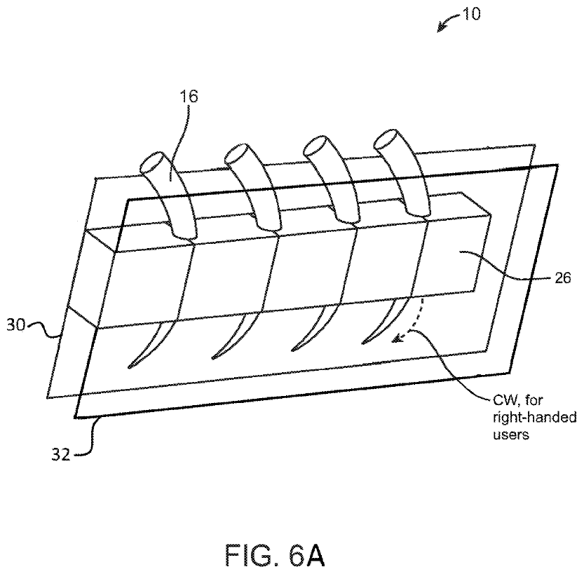

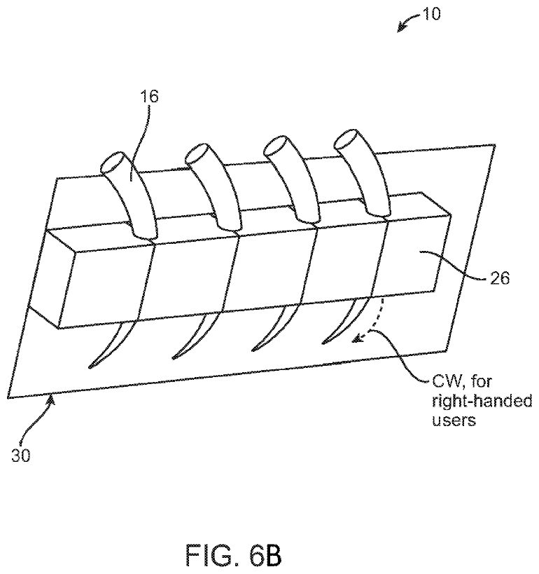

FIG. 6A illustrates adhesive covers 30, 32 on both sides of a suture needle package 10 in accordance with some embodiments. As described elsewhere herein, one or more adhesive removable covers 30, 32 can be provided on one or more sides of the housing 28. The covers 30, 32 can be applied onto the support 26 (e.g. which may be made of foam). In the example of FIG. 6A, the needles 16 are shown in the clockwise (CW) orientation for right handed use. In FIG. 6B shows the cover 32 removed for access in the CW orientation for right handed use, although the opposing cover could have been removed for left handed use in the CCW configuration. The covers 30, 32 can be transparent or obscuring.

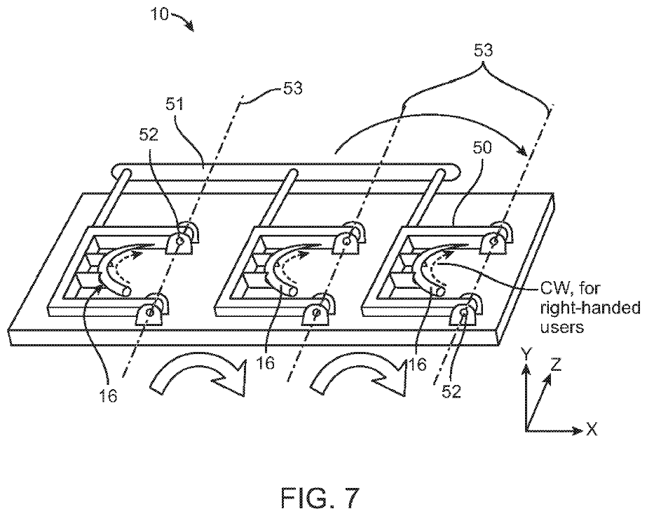

FIG. 7 illustrates a suture needle package 10 comprising a linkage mechanism 51 that is configured to simultaneously switch (rotate) a plurality of needles 16 between a first chiral configuration and a second chiral configuration. Each needle 16 may be coupled to a clip assembly 50 comprising a hinge 52 that is accessible from both sides (first and second sides) of the package. The linkage mechanism 51 can be coupled to a plurality of clip assemblies 50 for actuating a plurality of needles 16. Movement of the linkage 51 in the direction shown can cause the linkage 51 to engage and actuate the clip assemblies 50, and rotate the needles 16 to cause a change in orientation of the needles 16.

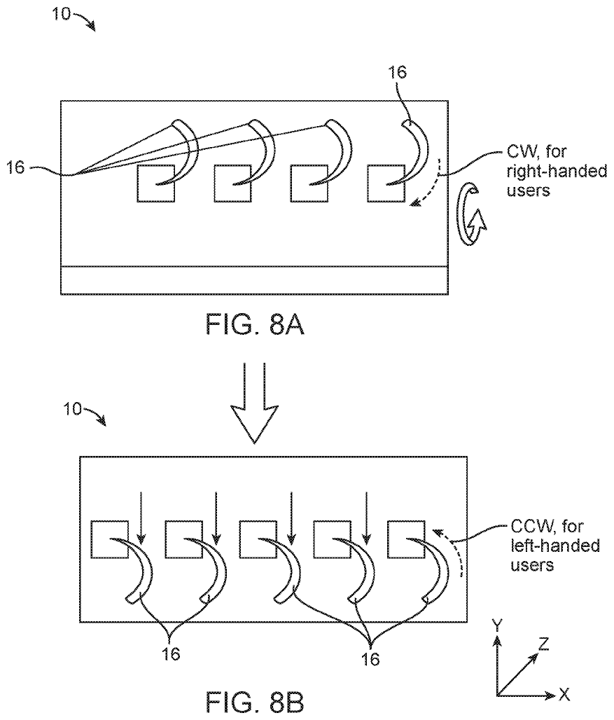

When the linkage mechanism 51 is moved in the direction as sh [symbol] mechanism 51 simultaneously engages the plurality of clip assemblies 50 and causes the needles 16 to rotate in a clockwise direction relative to the axis of rotation 53, thereby causing the needles 16 to move from the first orientation (clockwise CW) for right handed use to the second orientation (counter-clockwise CCW) for left handed use. Similarly, moving the linkage mechanism 51 in the opposite direction will result in the needles 16 moving from the second orientation (counter-clockwise CCW) for left handed use to the first orientation (clockwise CW) for right handed use. Accordingly, a plurality of needles 16 can be easily switched between different chiral configurations using the linkage mechanism 51. The batch rotation or simultaneous rotation of needles 16 is further illustrated in FIGS. 8A and 8B. FIG. 8A illustrates a suture needle package 10 comprising a plurality of needles 16 in a first chiral configuration for right-handed use. FIG. 8B shows the plurality of needles 16 rotated to a second chiral configuration for left-handed use in accordance with some embodiments.

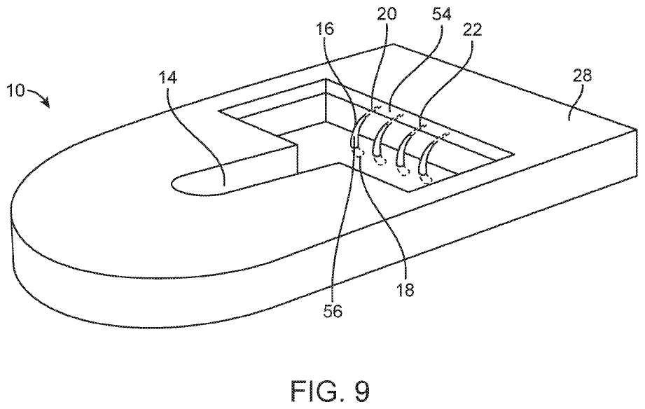

FIG. 9 illustrates a suture needle package 10 comprising a plurality of needles 16 that are configured for use from a user's perspective. The suture needle package 10 can be configured for both left handed and right-handed use. The suture needle package 10 can be agnostic to the user's master hand, since the package 10 is configured for use from the user's perspective. As shown in FIG. 9, the suture needle package 10 can comprise a needle receptacle 14, and one or more suture receiving channels 54. Each of the plurality of needles 16 may be oriented with a sharp end 18 and a tailing suture end 20 facing away from the needle receptacle 14, with a middle portion 56 of each of the plurality of needles 16 facing toward the needle receptacle 14. Optionally in some embodiments, each of the plurality of needles 16 may be oriented with a sharp end 18 and a suture end 20 facing away from the needle receptacle 14. The plurality of needles 16 can be arranged in an array with a sharp end 18 of each of the plurality of needles 16 extending into a support material. The support material may comprise, for example foam. When the needles 16 are ready to be used, the user can pull out the needles 16 from the support material, and dispense or manipulate the needles 16 in either a clockwise orientation (for right handed use) or counter-clockwise orientation (for left handed use). The tailing sutures 22 can be provided within one or more suture receiving channel(s) 54 facing towards the needle receptacle 14. In some embodiments, the one or more suture receiving channel(s) 54 may be provided along a chamfered edge of the housing 28, for example as shown in FIG. 9.

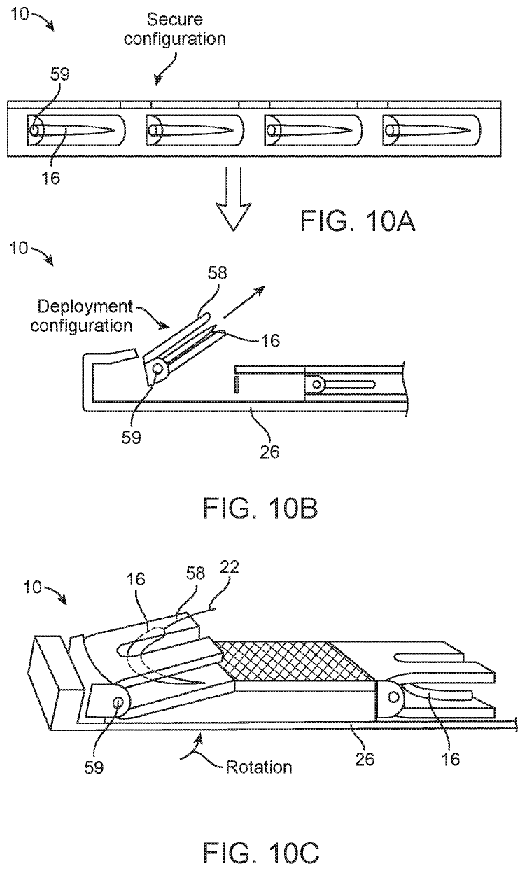

FIG. 10A illustrates a suture needle package 10 with clips 58 for holding suture needles 16 in a secure configuration. FIGS. 10B and 10C illustrate the suture needle package 10 of FIG. 10B with a clip 58 in a deployment configuration. The secure configuration may [symbol] configuration when the package 10 is not in use. The deployment configuration may correspond to a use configuration prior to use or when the package 10 is in use. The suture package 10 can comprise a clip 58 configured to hold a suture needle 16. The clip 58 can be configured to fold outward from the support 26 to allow the needle 16 to be grasped and removed from the clip 58 and subsequently placed in the clip 58. The clip 58 can also be configured to fold inward toward the support 26 to secure the needle 16. In some optional embodiments, the clip 58 can be folded outward toward a first side for right handed use or rotated outward toward a second side to allow for left handed use. The clip 58 can be configured to rotate about the rotational axis 59 shown in FIGS. 10A-10C. The clip 58 can be configured to rotate upward/outward by any amount, for example by 10 degrees, 20 degrees, 30 degrees, 40 degrees, 50 degrees or more than 50 degrees, or any value(s) therebetween.

Referring to FIGS. 10A-10C, an exemplary method of use may include one or more of the following steps: (1) grasping the needle 16 in a slot, (2) pulling up the clip 58 to unlock, (3) sliding the needle 16 out with the suture 22, (4) securing the needle 16 in the needle receptacle 14 or trap, and (5) pushing down the clip 58 to lock.

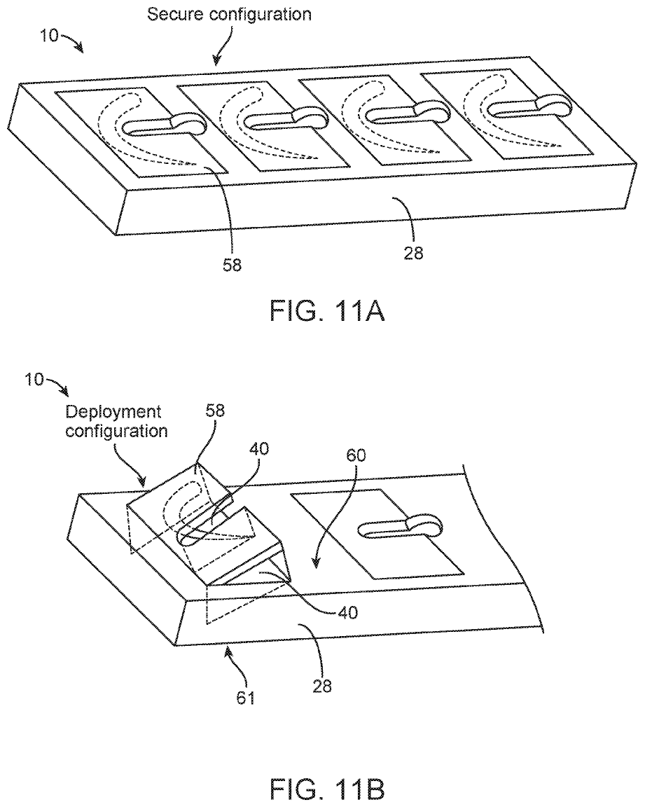

FIG. 11A illustrates a suture needle package 10 with clips 58 for holding suture needles in a secure configuration, and comprising needle drive slots 40. FIG. 11B illustrates the suture needle package 10 of FIG. 11A with a clip 58 in a deployment configuration with a needle drive slot 40 exposed. Examples of needle driver slots 40 are described in U.S. Pat. No. 9,826,975 entitled “Systems and methods for increased operating room efficiency,” which is incorporated herein by reference. In the example of FIGS. 11A and 11B, the trap (needle) dispenser may comprise a needle driver slot 40 and a needle slot 46. The housing 28 may comprise a landing zone 60 for the needles 16. The needle dispenser 24 and a needle receptacle 14 can be stored in a shared compartment 61 within the sterile housing 28. The needle dispenser 24 and needle receptacle 14 can be accessed from a same side. Optionally in some embodiments, a cover can be removed to expose the compartment to access the needle dispenser 24 and the needle receptacle 14 from the same side. In some cases, the cover can comprise a single cover.

In some embodiments, a chirality configurable suture package 10 can comprise a housing 28 to contain a plurality of curved suture needles 16 in a sterile configuration. The housing 28 can comprise a removable cover 30 on at least a first side of the suture package, and a support 26 to support the plurality of needles 16 within the housing 28. One or more of the support 26 or the housing 28 can be configured to allow the needles 16 to be grasped in a [symbol] to right handed use or a second chirality corresponding to left handed use.

An exemplary method in accordance with the present disclosure can comprise, determining whether a surgeon is left or right handed and configuring a suture dispensing package 10 and a needle receptacle 14 for the handedness of the surgeon. The suture dispensing package 10 and needle receptacle 14 may include any one or more of the embodiments described elsewhere herein.

In any of the embodiments described herein, the right-handed orientation may correspond to a clockwise orientation of the needle 16 when accessed by the needle driver 42, and the right-handed orientation may correspond to a counter clockwise orientation of the needle 16 when accessed by the needle driver 42. An orientation may correspond to a direction the needle is pointing with the sharp tip about an axis of rotation corresponding to a center of a radius of curvature of the curved needle 16. With the right-handed clockwise orientation, the needle tip 18 can point in a clockwise direction about the center of rotation if the needle is advanced with the tip 18 moving forward along a circumferential path defined by a center of rotation of the needle 16. With the left handed counter clockwise orientation, the needle tip 18 can point in a counter clockwise direction about the center of rotation as the needle 16 if the needle advanced with the tip 18 moving forward along a circumferential path defined by the center of curvature of the needle 16.

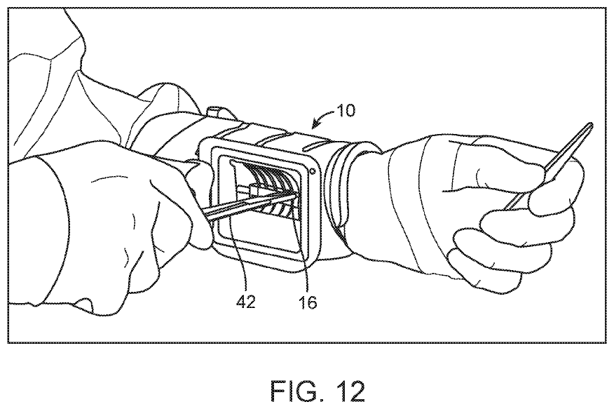

FIG. 12 illustrates a user such as a surgeon removing a curved suture needle 16 from a chiral suture needle package 10 and grasping the needle in a CW configuration as described herein. The curved suture needles 16 in this example are orientated in a chirality suitable for use by a right-handed user, e.g. the CW configuration. The user grasps the curved suture needle 16 and removes the curved suture needle 16 from the suture needle package 10. For example, the user may use a needle driver 42 to grasp the curved suture needle 16 and remove it from the suture needle package 10. The curved suture needle 16 is then secured by the needle driver 42 in a chiral configuration, e.g. CW, suitable for advancing the curved suture needle 16 into a working surface for stitching, depending on whether the user is left or right handed. The working surface may comprise an exposed skin of a patient with an incision to be closed with the suture needle, for example. Although reference is made to a CW and right-handed configuration, the method can be performed similarly for a left handed used with a counter clockwise (“CCW”) configuration is described herein.

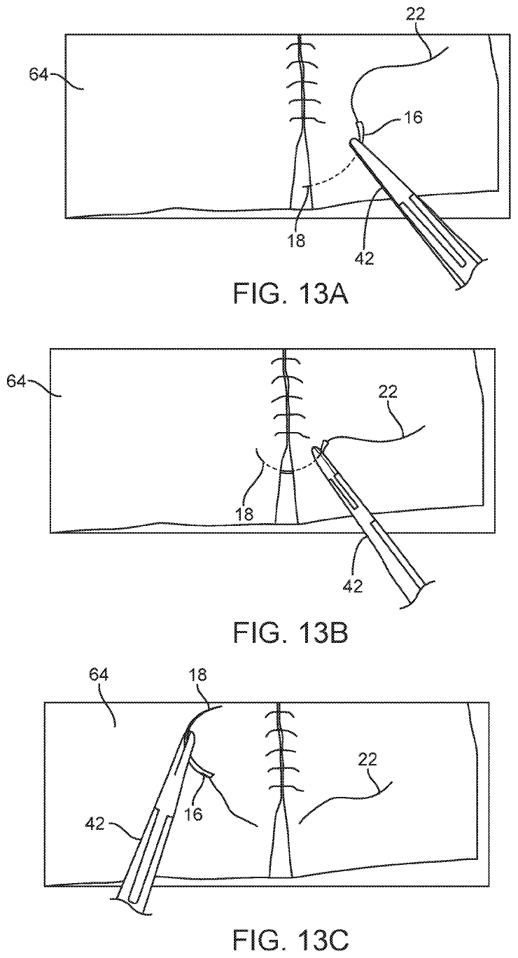

FIG. 13A through FIG. 13C illustrate a user advancing the curved suture needle 16 into a working surface 64 for stitching with suture 22. In FIG. 13A, a needle driver 42 has advanced the curved suture needle 16 into the working surface 64 such that a portion of the curved suture needle 16 including the sharp tip 18 is below the working surface 64. In FIG. 13 B, the needle driver 42 has further advanced the suture needle 16 into the working surface 64 with [symbol] sharp tip 18 of the curved suture needle 16 has curved back through the working surface 64 such that a portion of the curved suture needle 16 including the sharp tip 18 protrudes from the working surface 64. After the portion of the curved suture needle 16 including the sharp tip 18 protrudes from the working surface 64 the user may release the curved suture needle 16 from the needle driver 42. As illustrated in FIG. 13C, the user may then grasp the portion of the curved suture needle 16 that includes the sharp tip 18 and protrudes from the working surface 64, e.g. the skin of the patient. The user then removes the curved suture needle 16 from the working surface in the CW configuration.

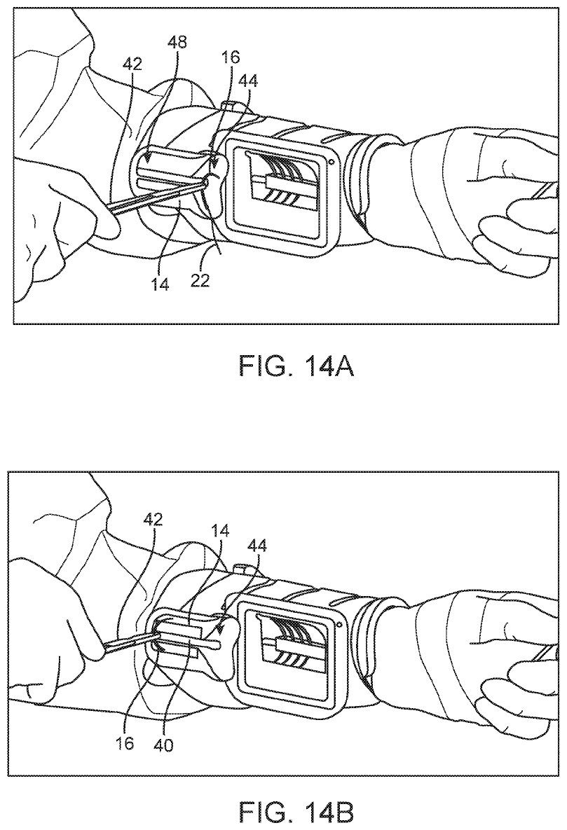

FIGS. 14A and 14B illustrate the curved suture needle 16 being returned to a needle receptacle 14 in the CW configuration. FIG. 14A illustrates a user using a needle driver 42 holding a curved suture needle to move the curved suture needle 16 into an entry zone 44 of the needle receptacle 14. The entry zone 44 can guide the curved needle 16 and/or the needle driver 42 into a secure zone 48 of the needle receptacle 14 for storage of the used curved suture needle 16 in the CW configuration. FIG. 14B illustrates the user having advanced the curved suture needle 16 to the secure zone 48 with needle driver 42 in the CW configuration. Needle driver 42 extends through a needle driver slot 40 to advance the curved suture needle 16 through the receptacle 14. When the curved suture needle 16 is in position in the secure zone 48, the user may release the curved suture needle 16 in the CW configuration and remove the needle driver 42 from the needle driver slot 40.

Although FIGS. 12 to 14B show a method of suturing in accordance with some embodiments, many alterations can be made. For example, the steps can be performed in a different order, some of the steps removed, and some of the steps repeated. Further, although reference is made to using sutures with a CW needle configuration for a right-handed user, the method can also be used with a CCW configuration for a left-handed user as described herein. By allowing the user such as a surgeon to grasp the needle with a needle driver with the appropriate chiral configuration from the needle package and then place the needle in the receptacle with the same chiral configuration, fewer manipulations of the needle are performed, resulting ease of use and in improved efficiency.

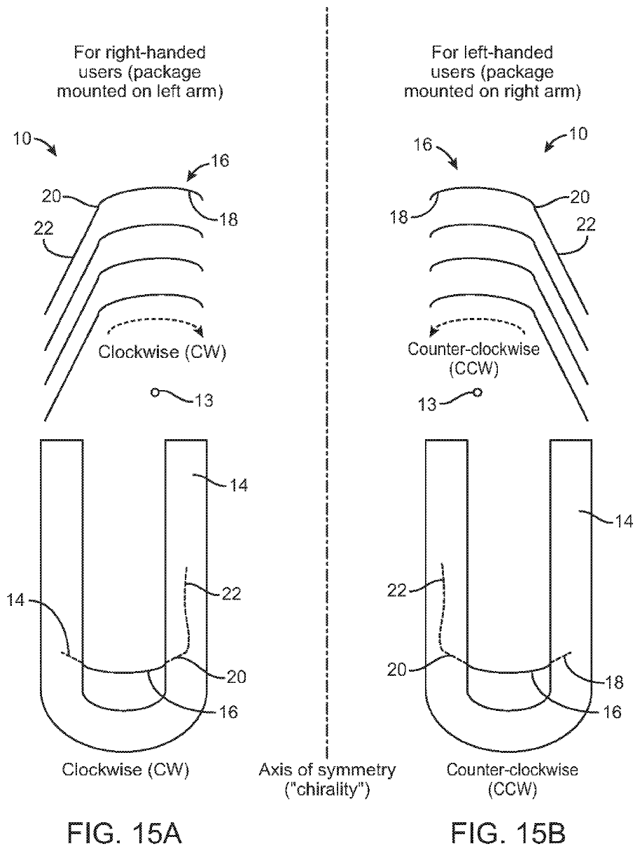

FIGS. 15A and 15B shows the orientation of chiral suture needles in a needle package and when placed in a needle receptacle to maintain chirality and decrease manipulations of the suture needle. The suture package 10 can be configured for both left-handed and right-handed use. For example, FIG. 15A shows a schematic of the suture needle package 10 in a first chiral configuration for right handed use, and FIG. 15B shows a schematic of the suture needle package 10 in a second chiral configuration for left handed use. The dotted line shows an axis of symmetry 12 (“chirality”) for the two chiral configurations.

Referring to FIG. 15A, the suture package 10 may comprise a n [symbol] plurality of suture needles 16 contained in a sterile suture needle package as described herein. A needle 16 may comprise a curved needle having a sharp tip 18 and a tail portions 20. The curved needle 16 may extend from the tip 18 to the tail portions 20. A suture 22 can be coupled to the tail portion 20 of the needle 16. The suture package 10 may be mounted on a left forearm of a user, with the tips 18 of the needles 16 extending away from the left forearm of the user. As shown in FIG. 15A, the needles 16 may be configured to move in a clockwise (CW) orientation about a center of reference axis for right handed use (for right handed users). A needle receptacle 14 may be coupled to the suture package 10 and configured to receive the suture needles 16 when the needles are in the first chiral configuration for right handed use, for example. The chiral configuration of the needles can be maintained when the suture needle has been placed in the receptacle. The needle comprises a clockwise (CW) orientation in the chiral needle package and when placed in the needle receptacle.

The suture package comprises a first plurality of curved suture needles comprising a tip portion and a tail portion and a curved portion extending between the tip portion and the tail portion with the tip portion pointing in a first direction corresponding to a CW orientation. The needle receptacle may comprise a second needle or a second plurality of needles removed from the suture package. The needle or second plurality of needles comprises a tip portion and a tail portion and a curved portion extending between the tip portion and the tail portion, the tip portion pointing in a second direction opposite the first direction.

Referring to FIG. 15B, the suture package 10 may be flipped relative to the axis of symmetry 12 such that the needles 16 are configured to move in a counter-clockwise (CCW) orientation about a center of reference axis for left handed use (for left handed users). The suture package 10 may be mounted on a right forearm of a user, with the tips 18 of the needles 16 extending away from the right forearm of the user. The needle receptacle 14 may be coupled to the suture package 10 and configured to receive the suture needles 16 when the needles are in the second chiral configuration for left handed use.

The suture package comprises a first plurality of curved suture needles comprising a tip portion and a tail portion and a curved portion extending between the tip portion and the tail portion with the tip portion pointing in a first direction corresponding to a counter clockwise (“CCW”) orientation. The needle receptacle may comprise a second needle or a second plurality of needles removed from the suture package. The needle or second plurality of needles comprises a tip portion and a tail portion and a curved portion extending between the tip portion and the tail portion, the tip portion pointing in a second direction opposite the first direction.

The needle package and needle receptacle can be passed from th [symbol] received by an assistant. Alternatively or in combination, the needle package and trap can be connected to each other and passed together as a unit after removal from the support on the forearm of the user, e.g. surgeon.

Although certain embodiments and examples are provided in the foregoing description, the inventive subject matter extends beyond the specifically disclosed embodiments to other alternative embodiments and/or uses, and to modifications and equivalents thereof. For example, the chiral needle packages and receptacles as described herein can be applied or used on any type of surgical devices. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein A and/or B encompasses one or more of A or B, and combinations thereof such as A and B. It will be understood that although the terms “first,” “second,” “third” etc. may be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to limit the present disclosure. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of sta [symbol] integers, steps, operations, elements and/or components, but do not preclude the presence or addition of one or more other features, regions, integers, steps, operations, elements, components and/or groups thereof.

Furthermore, relative terms, such as “lower” or “bottom” and “upper” or “top” may be used herein to describe one element's relationship to other elements as illustrated in the figures. It will be understood that relative terms are intended to encompass different orientations of the elements in addition to the orientation depicted in the figures. For example, if the element in one of the figures is turned over, elements described as being on the “lower” side of other elements would then be oriented on the “upper” side of the other elements. The exemplary term “lower” can, therefore, encompass both an orientation of “lower” and “upper,” depending upon the particular orientation of the figure. Similarly, if the element in one of the figures were turned over, elements described as “below” or “beneath” other elements would then be oriented “above” the other elements. The exemplary terms “below” or “beneath” can, therefore, encompass both an orientation of above and below.

This disclosure also includes the following numbered clauses.

Clause 1 An apparatus, comprising:
a needle dispenser configured to dispense one or more curved needles;
a needle receptacle to receive the one or more curved needles;
a support coupled to the needle receptacle and the needle dispenser; and
a housing to contain a plurality of curved suture needles in a sterile configuration, the housing comprising a removable cover on at least a first side of the housing;
wherein one or more of the support or the housing is configured to allow the needle to be grasped in a first chirality corresponding to right handed use or a second chirality corresponding to left handed use and the receptacle is configured to receive the needle with the needle dispenser in the first configuration or the second configuration.

Clause 2. The apparatus of clause 1, wherein the housing comprises a first removable cover on a first side for right handed access to the plurality of needles and the needle receptacle and a second removable cover on a second side for left handed access to the plurality of needles and the needle receptacle, the second side opposite the first side.

Clause 3. The apparatus of any one of the preceding clauses, wherein the housing comprises one or more markers indicating a quantity of one of more needles stored in the needle dispenser.

Clause 4. The apparatus of any one of the preceding clauses, wh [symbol] cover obscures a first marker and the second removable cover obscures a second marker.

Clause 5. The apparatus of any one of the preceding clauses, wherein the support is configured to move relative to the housing to rotate the needle from a first orientation corresponding to right handed access to a second orientation corresponding to left handed access and optionally wherein the needle receptacle comprises a substantially symmetric configuration to allow left handed use or right handed use when the needle comprises the first orientation or the second orientation.

Clause 6. The apparatus of any one of the preceding clauses, wherein the suture package comprises a clip configured to hold a suture needle, the clip configured to fold outward from the support to allow the needle to be grasped and removed from the clip and subsequently placed in the clip and wherein the clip is configured to fold inward toward the support to secure the needle and optionally wherein the clip can be folded outward toward a first side for right handed use or rotated outward toward a second side to allow for left handed use.

Clause 7. The apparatus of any one of the preceding clauses, wherein the support comprises a first holding structure to hold a curved needle toward a sharp tip end and a second holding structure to hold the needle toward a tailing suture end and an engagement structure to engage the needle between the first holding structure and the second holding structure in order to rotate the needle about an axis extending between the first holding structure and the second holding structure so as to change an orientation of the curved suture needle from a first orientation for right handed use to a second orientation for left handed use or vice versa.

Clause 8. The apparatus of any one of the preceding clauses, further comprising a needle receptacle, the needle receptacle coupled to the suture package and configured to receive the suture needles when the support or the housing comprises the first configuration for right handed use or the second configuration for left handed use.

Clause 9. The apparatus of any one of the preceding clauses, wherein the suture package and the needle receptacle are contained within the housing and supported together, and wherein the housing comprise a first removable cover on a first side for right handed access to the plurality of needles and the needle receptacle and a second removable cover on a second side for left handed access to the plurality of needles and the needle receptacle, the second side opposite the first side.

Clause 10. The apparatus of any one of the preceding clauses, wherein the needle receptacle comprises an entry zone and a needle slot to receive the needle, the entry zone sized larger than the needle slot to guide the needle into the needle slot, the needle receptacle comprising a needle driver slot to receive the needle driver and allow the needle driver to advance [symbol] zone and into the slot.

Clause 11. The apparatus of any one of the preceding clauses, wherein the plurality of needles is arrange in an array with a sharp end of each of the plurality of needles extending into a support material and optionally wherein the support material comprises foam and optionally wherein each of the plurality of needles is oriented with a sharp end and a tailing suture end facing away from the needle receptacle with a middle portion of said each of the plurality of needles facing toward the needle receptacle

Clause 12. The apparatus of any one of the preceding clauses, wherein the needle dispenser and the needle receptacle are stored in a shared compartment within the sterile housing, and wherein the needle dispenser and needle receptacle are accessed from a same side and optionally wherein a cover and be removed to expose the compartment to access the needle dispenser and the needle receptacle from the same side and optionally wherein the cover comprises a single cover.

Clause 13. The apparatus of any one of the preceding clauses, wherein the needle dispenser stores one or more needles with a sharp end orientated in a first direction and the needle receptacle is configured to receive a needle with the sharp end orientated in a second direction opposite the first direction.

Clause 14. A chirality configurable suture package, comprising:
a housing to contain a plurality of curved suture needles in a sterile configuration, the housing comprising a removable cover on at least a first side of the suture package; and
a support to support the plurality of needles within the housing;
wherein one or more of the support or the housing is configured to allow the needles to be grasped in a first chirality corresponding to right handed use or a second chirality corresponding to left handed use.

Clause 15. The suture package or apparatus of any one of the preceding clauses, wherein the housing comprises a first removable cover on a first side for right handed access to the plurality of needles and a second removable cover on a second side for left handed access to the plurality of needles, the second side opposite the first side.

Clause 16. The suture package or apparatus of any one of the preceding clauses, the suture package of claim 1, wherein the support is configured to move relative to the housing to rotate the needle from a first orientation corresponding to right handed access to a second orientation corresponding to left handed access.

Clause 17. The suture package or apparatus of any one of the pr [symbol] suture package comprises a clip configure to hold a suture needle, the clip configured to fold outward from the support to allow the needle to be grasped and removed from the clip and subsequently placed in the clip, and the clip configured to fold inward toward the support to secure the needle.

Clause 18. The suture package or apparatus of any one of the preceding clauses, wherein the support comprises a first holding structure to hold a curved needle toward a sharp tip end and a second holding structure to hold the needle toward a suture end and an engagement structure to engage the needle between the first holding structure and the second holding structure in order to rotate the needle about an axis extending between the first holding structure and the second holding structure so as to change an orientation of the curved suture needle from a first orientation for right handed use to a second orientation for left handed use.

Clause 19. The suture package or apparatus of any one of the preceding clauses, further comprising a needle receptacle, the needle receptacle coupled to the suture package and configured to receive the suture needles when the support or the housing comprises the first configuration for right handed use or the second configuration for left handed use.

Clause 20. The suture package or apparatus of any one of the preceding clauses, wherein the suture package and the needle receptacle are contained within the housing and supported together, and wherein the housing comprise a first removable cover on a first side for right handed access to the plurality of needles and the needle receptacle and a second removable cover on a second side for left handed access to the plurality of needles and the needle receptacle, the second side opposite the first side.

Clause 21. The suture package or apparatus of any one of the preceding clauses, wherein the needle receptacle comprises an entry zone and a needle slot to receive the needle, the entry zone sized larger than the needle slot to guide the needle into the needle slot, the needle receptacle comprising a needle driver slot to receive the needle driver and allow the needle driver to advance the needle along the entry zone and into the slot.

Clause 22. The suture package or apparatus of any one of the preceding clauses, wherein the plurality of needles is arranged in an array with a sharp end of each of the plurality of needles extending into a support material and optionally wherein the support material comprises foam and optionally wherein each of the plurality of needles is oriented with a sharp end and a suture end facing away from the needle receptacle.

Clause 23. A method comprising, determining whether a surgeo [symbol] configuring a suture dispensing package and a needle receptacle for the handedness of the surgeon.

Clause 24. The apparatus, suture package or method of any one of the preceding claims, wherein:
the suture package comprises a first plurality of curved suture needles comprising a tip portion and a tail portion and a curved portion extending between the tip portion and the tail portion, the tip portion pointing in a first direction;
the needle receptacle comprises a second plurality of needles removed from the suture package, the second plurality of needles comprising a tip portion and a tail portion and a curved portion extending between the tip portion and the tail portion, the tip portion pointing in a second direction opposite the first direction.

Clause 25. The apparatus, suture package or method any one of the preceding clauses, wherein the suture package comprises a marker indicating a number of suture needles contained in the suture package prior to removal of a cover of the suture package and optionally wherein the marker comprises a first marker on a first side of the suture package and a second marker on a second side of the suture package, each of said markers indicating the number of suture needles contained in the suture package prior to removal of a sterile cover.

Clause 26. A method of handing a suture needle, comprising:
grasping a curved suture needle secured in a chirality configurable suture package, wherein the suture needle comprises a sharp tip portion and a tail portion connected to a suture, a curved portion extending between the tip portion and the tail portion corresponding to a chiral configuration of the curved suture needle in the suture package;
advancing the curved suture needle into a working surface until the tip portion of the curved suture needle protrudes from the working surface;
releasing the suture needle after the tip portion of the curved suture needle protrudes form the working surface;
grasping the tip portion of the curved suture needle protruding from the working surface; and
inserting the curved suture needle into a needle receptacle with an orientation corresponding to the chiral configuration.

Clause 27. The suture package, method or apparatus of any one of the preceding clauses, wherein the suture needle is oriented in a first direction in the suture package and the suture needle is oriented in a second direction in the suture package, the second direction opposite the first direction and the curve portion of the needle in the suture package oriented oppo [symbol] suture needle in the needle receptacle in order to maintain the chiral configuration.

Clause 28. The suture package, method or apparatus of any one of the preceding clauses, wherein the needle in the suture package points upwardly and outwardly from a midline of the user with the curved portion facing anteriorly away from the user, and wherein the needle placed in the receptacle points downwardly and inwardly with the curved portion of the needle facing posteriorly relative to the user and optionally wherein the user comprises a surgeon.

Clause 29. The suture package, method or apparatus of any one of the preceding clauses wherein the needle comprises a curved needle extending from a sharp tip on a first end to a suture on a second end and wherein the right handed orientation corresponds to a clockwise orientation of the needle when accessed by the needle driver and the left handed orientation corresponds to a counter clockwise orientation of the needle when accessed by the needle driver and optionally wherein the orientation corresponds to a direction the needle is pointing with the sharp tip about an axis of rotation corresponding to a center of a radius of curvature of the curved needle and optionally wherein with the right handed clockwise orientation the needle tip points in a clockwise direction about the center of rotation if the needle is advanced with the tip moving forward along a circumferential path defined by a center of rotation of the needle and optionally wherein with the left handed counter clockwise orientation the needle tip points in a counter clockwise direction about the center of rotation as the needle if the needle advanced with the tip moving forward along a circumferential path defined by the center of curvature of the needle.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the described embodiments will be apparent to a person skilled in the art. It is therefore contemplated that the invention shall also cover any such modifications, variations and equivalents.

1. An apparatus, comprising:
a needle dispenser configured to dispense one or more curved needles;
a needle receptacle to receive the one or more curved needles;
a support coupled to the needle receptacle and the needle dispenser; and
a housing to contain a plurality of curved suture needles in a sterile configuration, the housing comprising a removable cover on at least a first side of the housing;
wherein one or more of the support or the housing is configured to allow the needle to be grasped in a first chirality corresponding to right handed use or a second chirality corresponding to left handed use and the receptacle is configured to receive the needle with the needle dispenser in the first configuration or the second configuration.

2. The apparatus of claim 2, wherein the housing comprises a first removable cover on a first side for right handed access to the plurality of needles and the needle receptacle and a second removable cover on a second side for left handed access to the plurality of needles and the needle receptacle, the second side opposite the first side.

3. The apparatus of claim 2, wherein the housing comprises one or more markers indicating a quantity of one of more needles stored in the needle dispenser.

4. The apparatus of claim 3, wherein the first removable cover obscures a first marker and the second removable cover obscures a second marker.

5. The apparatus of claim 2, wherein the support is configured to move relative to the housing to rotate the needle from a first orientation corresponding to right handed access to a second orientation corresponding to left handed access.

6. The apparatus of claim 2, wherein the suture package comprises a clip configured to hold a suture needle, the clip configured to fold outward from the support to allow the needle to be grasped and removed from the clip and subsequently placed in the clip and wherein the clip is configured to fold inward toward the support to secure the needle.

7. The apparatus of claim 2, wherein the support comprises a first holding structure to hold a curved needle toward a sharp tip end and a second holding structure to hold the needle toward a tailing suture end and an engagement structure to engage the needle between the first holding structure and the second holding structure in order to rotate the needle about an axis extending between the first holding structure and the second holding structure so as to change an orientation of the curved suture needle from a first orientation for right handed use to a second orientation for left handed use or vice versa.

8. The apparatus of claim 2, further comprising a needle receptacle, the needle receptacle coupled to the suture package and configured to receive the suture needles when the support or the housing comprises the first configuration for right handed use or the second configuration for left handed use.

9. The apparatus of claim 8, wherein the suture package and the needle receptacle are contained within the housing and supported together, and wherein the housing comprise a first removable cover on a first side for right handed access to the plurality of needles and the needle receptacle and a second removable cover on a second side for left handed access to the plurality of needles and the needle receptacle, the second side opposite the first side.

10. The apparatus of claim 9, wherein the needle receptacle comprises an entry zone and a needle slot to receive the needle, the entry zone sized larger than the needle slot to guide the needle into the needle slot, the needle receptacle comprising a needle driver slot to receive the needle driver and allow the needle driver to advance the needle along the entry zone and into the slot.

11. The apparatus of claim 10, wherein the plurality of needles is arrange in an array with a sharp end of each of the plurality of needles extending into a support material.

12. The apparatus of claim 2, wherein the needle dispenser and the needle receptacle are stored in a shared compartment within the sterile housing, and wherein the needle dispenser and needle receptacle are accessed from a same side.

13. The apparatus of claim 8, wherein the needle dispenser stores one or more needles with a sharp end orientated in a first direction and the needle receptacle is configured to receive a needle with the sharp end orientated in a second direction opposite the first direction.

14. A chirality configurable suture package, comprising:
a housing to contain a plurality of curved suture needles in a sterile configuration, the housing comprising a removable cover on at least a first side of the suture package; and
a support to support the plurality of needles within the housing;
wherein one or more of the support or the housing is configured to allow the needles to be grasped in a first chirality corresponding to right handed use or a second chirality corresponding to left handed use.

15. The suture package of claim 14, wherein the housing comprises a first removable cover on a first side for right handed access to the plurality of needles and a second removable cover on a second side for left handed access to the plurality of needles, the second side opposite the first side.

16. The suture package of claim 14, the suture package of claim 1, wherein the support is configured to move relative to the housing to rotate the needle from a first orientation corresponding to right handed access to a second orientation corresponding to left handed access.

17. The suture package of claim 14, wherein the suture package comprises a clip configured to hold a suture needle, the clip configured to fold outward from the support to allow the needle to be grasped and removed from the clip and subsequently placed in the clip, and the clip configured to fold inward toward the support to secure the needle.

18. The suture package of claim 14, wherein the support comprises a first holding structure to hold a curved needle toward a sharp tip end and a second holding structure to hold the needle toward a suture end and an engagement structure to engage the needle between the first holding structure and the second holding structure in order to rotate the needle about an axis extending between the first holding structure and the second holding structure so as to change an orientation of the curved suture needle from a first orientation for right handed use to a second orientation for left handed use.

19. The suture package of claim 14, further comprising a needle receptacle, the needle receptacle coupled to the suture package and configured to receive the suture needles when the support or the housing comprises the first configuration for right handed use or the second configuration for left handed use.

20. The suture package of claim 19, wherein the suture package and the needle receptacle are contained within the housing and supported together, and wherein the housing comprise a first removable cover on a first side for right handed access to the plurality of needles and the needle receptacle and a second removable cover on a second side for left handed access to the plurality of needles and the needle receptacle, the second side opposite the first side.

21. The suture package of claim 20, wherein the needle receptacle comprises an entry zone and a needle slot to receive the needle, the entry zone sized larger than the needle slot to guide the needle into the needle slot, the needle receptacle comprising a needle driver slot to receive the needle driver and allow the needle driver to advance the needle along the entry zone and into the slot.

22. The suture package of claim 21, wherein the plurality of needles is arranged in an array with a sharp end of each of the plurality of needles extending into a support material.

23.-28. (canceled)

29. The apparatus of claim 1, wherein the needle comprises a curved needle extending from a sharp tip on a first end to a suture on a second end and wherein the right handed orientation corresponds to a clockwise orientation of the needle when accessed by the needle driver and the left handed orientation corresponds to a counter clockwise orientation of the needle when accessed by the needle driver.
